(19) United States
(12) Patent Application Publication
(10) Pub. No.: US 2018/0042672 A1
(43) Pub. Date: Feb. 15, 2018

(54) ELECTRODE ASSEMBLY HAVING ASYMMETRIC ELECTRODE PLACEMENT

(71) Applicant: St. Jude Medical, Cardiology Division, Inc., St. Paul, MN (US)

(72) Inventors: Jennifer Marie Heisel, Princeton, MN (US); Paul Aaron Belk, Maple Grove, MN (US)

(21) Appl. No.: 15/790,468

(22) Filed: Oct. 23, 2017

Related U.S. Application Data
(62) Division of application No. 14/512,505, filed on Oct. 13, 2014.
(60) Provisional application No. 61/895,181, filed on Oct. 24, 2013.

(51) Int. Cl. A61B 18/14; A61B 18/00

(52) U.S. Cl. CPC A61B 18/1492; A61B 2018/1467; A61B 2018/00577; A61B 2018/00267; A61B 2018/00434; A61B 2018/00404; A61B 2018/00511

(57) ABSTRACT

The present disclosure provides ablation catheter systems and electrode assemblies, including electrode baskets, for use in the ablation catheter systems for ablating tissue. The electrode baskets include a plurality of struts having a plurality of electrodes asymmetrically arranged on the plurality of struts such that upon simultaneous energizing of the plurality of electrodes during any single application of the ablation procedure, lesions are created on only one half of less of the circumference of the vessel.

### CROSS-REFERENCE TO RELATED APPLICATIONS

This application claims priority to provisional application Ser. No. 61/895,181, filed Oct. 24, 2013, the entire specification of which is incorporated herein.

### FIELD OF THE DISCLOSURE

The present disclosure relates generally to an electrode assembly for use in a human body. More particularly the present disclosure relates to an electrode basket having asymmetric electrode placement on the electrode basket struts and related uses.

### Background Art

Catheter systems are well known in the art for use in medical procedures, such as diagnostic, therapeutic and ablative procedures. Typical catheter systems generally include an elongate catheter extending from a handle. A physician manipulates the catheter through the patient's vasculature to an intended site within the patient. The catheter typically carries one or more working components, such as electrodes and thermocouples, or other diagnostic, therapeutic or ablative devices for carrying out the procedures. One or more controls or actuators may be provided on the handle for selectively adjusting one or more characteristics of the working components.

One particular example of a multi-electrode catheter system is an ablative catheter system in which the working component is a multi-electrode assembly carried at the distal end of a flexible catheter. A control wire generally extends within the catheter from the multi-electrode assembly to the handle to operatively connect the multi-electrode assembly to an actuator on the handle. Manipulating the actuator acts on the control wire to configure the multi-electrode assembly into a desired configuration for carrying out the ablative procedure. For example, in one such ablative catheter system made by St. Jude Medical, Inc. under the trade name EnligHTN, the multi-electrode assembly is an electrode assembly in the general form of an electrode basket. The electrode basket generally includes a number of nitinol struts, wherein each strut may include one or two electrodes for ablating vessel tissue. The electrode basket is formed using the nitinol struts in combination with an adhesive material that is used at certain points to hold the electrode basket together for use in an ablation procedure.

Once positioned at the intended site within the patient, such as a renal artery, the electrodes on the electrode basket may be simultaneously energized to ablate tissue about the circumference of the artery. After an ablation is complete, the electrode basket is generally pulled along the length of the artery and may optionally be rotated about the circumference of the artery (for example, 45 or 90 degrees) for any additional ablation procedure. The ablation procedure may include two, three, or more individual successive ablations of tissue.

### BRIEF SUMMARY OF THE DISCLOSURE

In one embodiment, the present disclosure is directed to an electrode basket for ablating vessel tissue. The electrode basket comprises a plurality of struts and a plurality of electrodes, wherein the plurality of electrodes are arranged on the plurality of struts such that the electrode basket is capable of ablating the vessel tissue upon simultaneous energizing of each electrode such that a contiguous angular region of less than 180 degrees of the vessel is ablated.

In another embodiment, the present disclosure is directed to a method of ablating vessel tissue. The method comprises introducing an electrode basket into the vessel, wherein the electrode basket comprises a plurality of struts and a plurality of electrodes, and simultaneously energizing the plurality of electrodes such that a contiguous angular region of less than 180 degrees of the vessel is ablated.

In another embodiment, the present disclosure is directed to an electrode basket for ablating tissue. The electrode basket comprises four struts, wherein only two adjacent struts include electrodes, such that the electrode basket is capable of ablating the vessel tissue upon simultaneous energizing of each electrode such that a contiguous angular region of less than 150 degrees of the vessel is ablated.

The foregoing and other aspects, features, details, utilities and advantages of the present disclosure will be apparent from reading the following description and claims, and from reviewing the accompanying drawings.

### BRIEF DESCRIPTION OF THE DRAWINGS

FIG. 1 is a perspective view of one embodiment of a catheter system including a handle, a catheter and an electrode assembly having multiple electrodes, with the electrode assembly being in a collapsed configuration.

FIG. 2 is a side elevation of the catheter system of FIG. 1, with the electrode assembly being in an expanded configuration resulting from rotation of a rotatable actuator.

FIG. 3 is an expanded view of a conventional electrode basket including electrodes placed on each strut.

FIG. 4 illustrates an electrode basket of the present disclosure including four struts and asymmetric electrode placement.

FIG. 5 illustrates an electrode basket of the present disclosure including six struts and asymmetric electrode placement.

FIG. 6 illustrates an electrode basket of the present disclosure including 2 struts and asymmetric electrode placement.

FIG. 7 illustrates an electrode basket of the present disclosure including 3 struts and asymmetric electrode placement.

FIG. 8 illustrates an electrode basket of the present disclosure including 5 struts and asymmetric electrode placement.

Corresponding reference characters indicate corresponding parts throughout the several views of the drawings.

### DETAILED DESCRIPTION OF THE DISCLOSURE

The present disclosure provides ablation catheter systems and electrode assemblies, including electrode baskets, for use in the ablation catheter systems for ablating tissue, and specifically tissue in a vessel, such as a renal artery. The present disclosure also provides methods of ablating vessel tissue using the electrode baskets described herein. The electrode baskets include a plurality of struts having a plurality of electrodes asymmetrically arranged on the plurality of struts such that upon simultaneous energizing of the plurality of electrodes during any single application of energy during an ablation procedure, lesions are created on only one half or less of the circumference of the vessel; that is, the electrodes are located on the struts of the electrode basket such that only a limited angular region of the vessel is affected by any one single application of the ablation procedure, as compared to ablation over substantially the entire circumference of the vessel during a single application of the ablation procedure. Many types of electrode baskets are suitable for use with the present disclosure, including electrode baskets that use balloons and electrode baskets with different shaped struts, including spiral struts. Many of the struts of the electrode baskets described herein will include one or more living hinges.

By limiting the angular region of the vessel that is ablated during a single application of energy during an ablation procedure such that lesions are created only on about one half or less of the circumference of the vessel, the potential for unintended merger of multiple lesions about the circumference of the vessel into a single, larger continuous lesion about the entire circumference of the vessel, such as continuous lesion merger during a vessel spasm during the ablation procedure, is significantly reduced or eliminated, which can result in improved patient outcomes. By utilizing the electrode baskets of the present disclosure including asymmetrically arranged electrodes, no single application of energy during an ablation procedure can result in a merged circumferential lesion on the vessel.

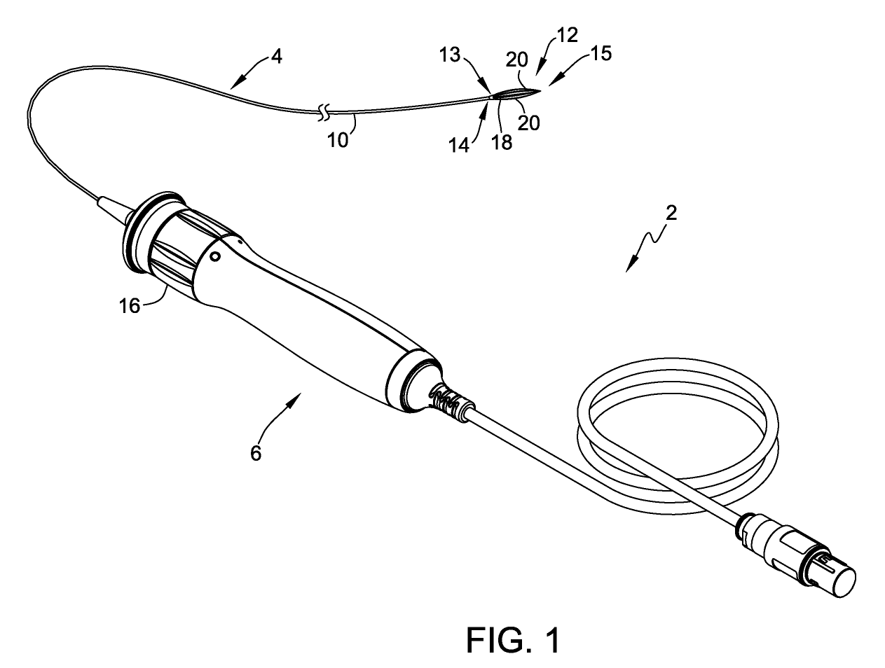

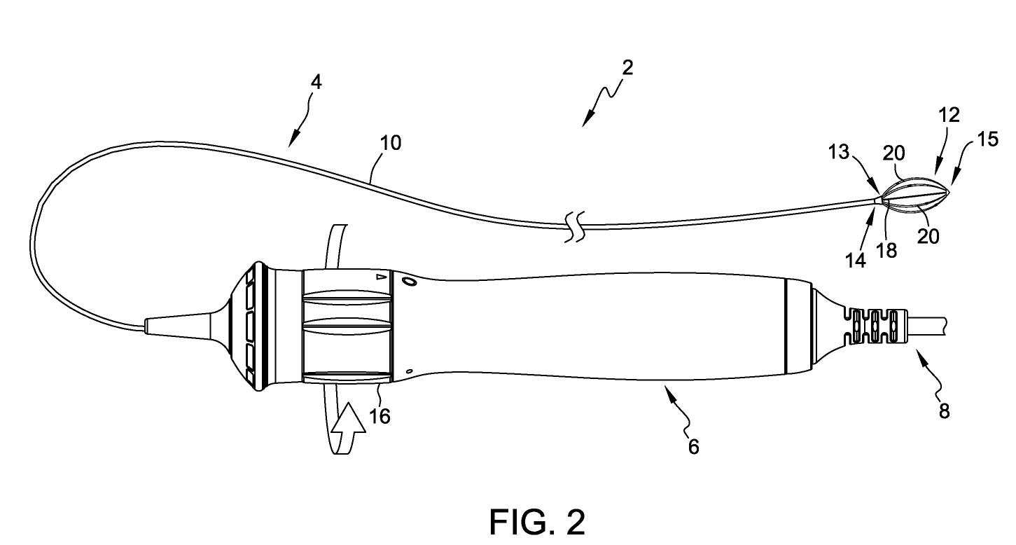

Referring now to the drawings, and in particular to FIGS. 1 and 2, a conventional catheter system 2 is shown by way of background and reference. Catheter system 2 includes a flexible catheter 4, a handle 6 to which flexible catheter 4 is connected, and a conductor assembly 8 for electrically connecting catheter system 2 to a suitable power supply (not shown). As one example, catheter system 2 illustrated and described herein is suitably constructed for use as an ablation system, such as a renal or heart ablation system. More particularly, illustrated catheter system 2 is a multi-electrode renal denervation system. One example of such a catheter system 2 is currently made by St. Jude Medical, Inc. under the trade name EnligHTN. General operation of a multi-electrode renal denervation system is known to those of skill in the art and is not described further herein except to the extent necessary to describe the present embodiments. It is also understood that catheter system 2 may be used for any other suitable treatment or purpose without departing from the scope of this disclosure. Additionally, while catheter system 2 is illustrated and described herein as including flexible catheter 4, catheter system 2 may further include other components used, for example, to guide flexible catheter 4 into the patient—such as, without limitation, a relatively more rigid guide catheter (not shown) or guide wire (not shown).

Flexible catheter 4 includes an elongate, flexible hollow shaft 10 connected to handle 6 at or near a proximal or rear end of the catheter shaft (not shown because it is hidden by a connector at the front end of handle 6), and an electrode assembly 12 disposed at or near a distal or front end 14 of flexible hollow shaft 10. Electrode assembly 12 includes proximal end 13 and distal end 15. It is understood, however, that electrode assembly 12 may be disposed anywhere along flexible hollow catheter shaft 10 intermediate the proximal end and the distal end 14 thereof without departing from the scope of this disclosure. As used herein, the terms proximal and front, and distal and rear, are used with reference to the orientation of catheter system 2 illustrated in the various drawings and for the purpose of describing the various embodiments set forth herein, and are not intended as limiting the catheter system and related components to having any particular orientation upon assembly or during operation thereof. In particular, the terms proximal and rear refer to a longitudinal position that is relatively nearer to handle 6 while the terms distal and front refer to a longitudinal position that is relatively farther from handle 6.

Illustrated electrode assembly 12 is in the form of what may be referred to as an electrode basket and includes struts 20, and is suitably configurable between a collapsed configuration (FIG. 1) for maneuvering and positioning the electrode assembly in the patient, and an expanded configuration (FIG. 2) for operation of the electrode assembly to perform a desired procedure such as an ablation procedure. An annular (e.g., ring-shaped) actuator 16 is mounted on handle 6 for rotation relative thereto and is operatively connected to electrode assembly 12 for selectively configuring the electrode assembly between its collapsed and expanded configurations. It is understood that another suitable actuator (e.g., slide, push button, lever, etc.) may be used instead of rotating actuator 16 to selectively configure electrode assembly 12 without departing from the scope of this disclosure. In some embodiments, electrode assembly 12 may be selectively adjustable between an infinite number of configurations (e.g., degrees of expansion) between its collapsed and expanded configurations using actuator 16.

A control line, such as a suitable cable or pull wire 18 extends from electrode assembly 12 within hollow catheter shaft 10 and into the handle 6 for operative connection with the actuator to thereby operatively connect the actuator 16 with electrode assembly 12. In some embodiments two or more pull wires, cables or other suitable control lines or tubes may be used for selectively configuring electrode assembly 12. It is also understood that control line 18 may be any suitable control line other than a pull wire, such as a cable, string, tie, compression member or other suitable control to operatively connect electrode assembly 12 to actuator 16, or that other means of force transduction, such as a column of fluid or gas or other means of compression, may be used to expand the basket. A suitable twisted electrical wire bundle (not shown) also extends through hollow catheter shaft 10 from handle 6 to electrode assembly 12 to deliver power to electrode assembly 12.

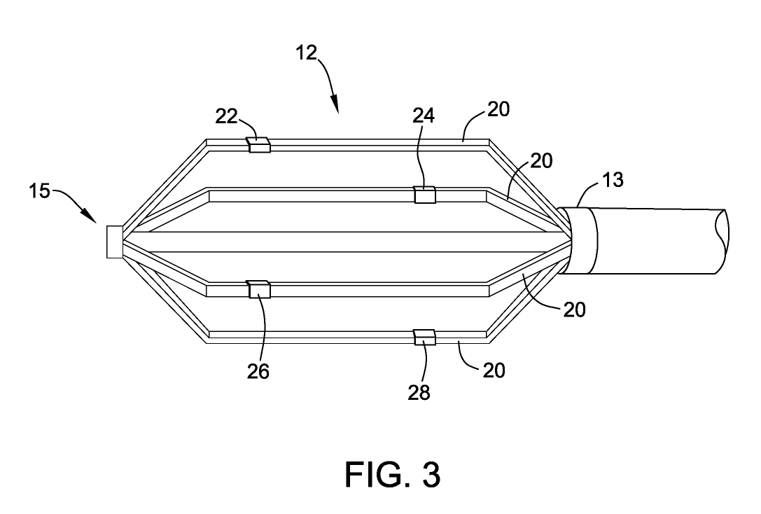

Referring now to FIG. 3, there is shown an expanded view of a conventional electrode assembly in an expanded configuration suitable for use in a conventional renal denervation procedure. Electrode assembly 12 includes proximal end 13, distal end 15, struts 20, and electrodes 22, 24, 26, and 28. In this conventional embodiment, each of struts 20 includes one electrode (22, 24, 26, or 28) disposed thereon. With this electrode arrangement on the struts, upon simultaneous energizing of the electrodes as is typical in a denervation procedure, a vessel receives ablation treatment about substantially its entire circumference.

As noted above, the present disclosure provides an electrode assembly, generally in the form of an electrode basket, that has one or more electrodes placed on a one or more struts forming the electrode basket in an asymmetric manner such that no single application of energy during an ablation procedure results in a circumferential treatment (formation of lesions) of the vessel. In accordance with the present disclosure, an electrode basket may include two strut, three struts, four struts, five struts, six struts or more than six struts and any strut may include one electrode, two electrodes, three electrodes or more. As used herein, the term “asymmetric electrode placement” refers to the placement of electrodes on the struts of an electrode basket in a manner such that upon energizing of all electrodes present simultaneously, less than then entire circumference of the vessel being treated is ablated. The struts may be comprised of any suitable material, including memory shape alloys such as nitinol.

In accordance with the present disclosure, the electrode baskets include a plurality of struts including a plurality of electrodes arranged thereon in such a manner that only a limited angular region of a vessel is ablated upon energizing of all of the electrodes present on the basket during a single application of the ablation procedure. Generally, less than one half of the total circumference of the vessel is ablated during a single application using the electrode baskets having asymmetric electrode placement; that is, upon a single application of energy during the ablation of the vessel utilizing the electrode baskets as described herein, a contiguous angular region of the vessel of less than 180 degrees is ablated. By limiting each individual ablation to a contiguous angular region of less than 180 degrees on the vessel, the likelihood of lesion merger to form a continuous lesion about the circumference of the vessel, such as continuous lesion merger during a vessel spasm, is eliminated, as noted above. That is, even if a vessel spasm occurs during an ablation procedure, because an angular region of less than 180 degrees on the vessel is being ablated, at most the lesions created from the current ablation could merge forming a larger ablation spanning a contiguous angular region of less than 180 degrees. In some instances, a spasm could be cause for discontinuing the ablation procedure, rather than continuing to a second lesion set, or for displacing the catheter along the length of the artery. In the absence of a spasm, however, the lesions created would be neither contiguous nor span 180 degrees.

As a second ablation, after rotating electrode basket 30 about 180 degrees from the position of the first ablation as described herein, would also span a contiguous angular region of less than 180 degrees, even in the event of another spasm and lesion merger during the second ablation, if the ablation procedure were to continue after a spasm during the first ablation, the lesions created by the first ablation and the lesions created by the second ablation would not be able to merge with one another to form a circumferential lesion about the vessel as there are not electrodes present about the entire circumference of the vessel.

In other embodiments in accordance with the present disclosure, a contiguous angular region of less than 175 degrees, or even 170 degrees, or even 160 degrees or even 150 degrees is ablated by the electrode basket as described herein. This is further discussed and numerous embodiments disclosed below.

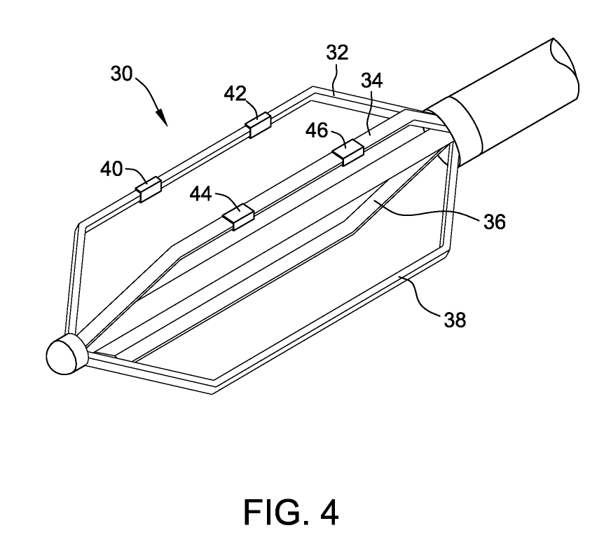

Referring now to FIG. 4, there is shown an electrode basket of the present disclosure including four struts and asymmetric electrode placement. FIG. 4 shows electrode basket 30 including struts 32, 34, 36, and 38. Strut 32 includes electrodes 40 and 42 and strut 34 includes electrodes 44 and 46. Struts 36 and 38 do not include electrodes in this embodiment such that the electrodes present are arranged asymmetrically. With this electrode arrangement on the struts of the electrode basket, upon insertion into a vessel and simultaneous energizing of the electrodes present on the struts, ablations from the four electrodes would occur over a contiguous angular region of the vessel of less than 180 degrees; that is, the four ablations would occur on the vessel on less than one half of the circumference of the vessel. Because there is not ablation of tissue around the entire, or substantially the entire, circumference of the vessel during a single application of energy during the ablation procedure, the merger of the lesions, upon a spasm of the vessel for example, cannot occur as there is a significant amount of untreated tissue along the circumference of the vessel. Additionally, if a total number of eight ablations is desired to meet the standard of care for the procedure, an electrode basket as illustrated in FIG. 4 can be rotated 180 degrees and the electrodes energized a second time to ablate an additional four locations on the artery. Because circumferential treatment of the artery did not occur with the first set of ablations, it is not required that the electrode basket be pulled lengthwise through the vessel prior to the second set of ablations. Again, the second set of ablations would occur over a contiguous angular region of the vessel of less than 180 degrees, which would prevent the merger of the lesions in the event of a vessel spasm.

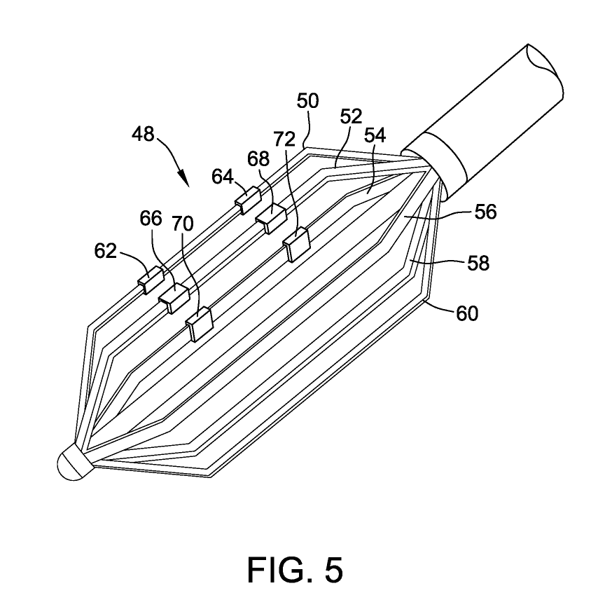

FIG. 5 illustrates another embodiment of the present disclosure and shows a six strut design and asymmetric electrode placement on the struts. FIG. 5 shows electrode basket 48 including struts 50, 52, 54, 56, 58, and 60. Strut 50 includes electrodes 62 and 64, strut 52 includes electrodes 66 and 68, and strut 54 includes electrodes 70 and 72. Struts 56, 58, and 60 do not include electrodes in this embodiment such that the electrodes present are arranged asymmetrically on the struts of the electrode basket. With this electrode arrangement on the struts of the electrode basket, upon insertion into a vessel and simultaneous energizing of the electrodes present on the struts, ablations from the six electrodes would occur over a contiguous angular region of the vessel of less than 180 degrees; that is, the six ablations would occur on the vessel on less than one half of the circumference of the vessel. Because there is not ablation of tissue around the entire, or substantially the entire, circumference of the vessel during a single application of energy during the ablation procedure, the merger of the ablations, upon a spasm of the vessel for example, cannot occur as there is a significant amount of untreated tissue along the circumference of the vessel.

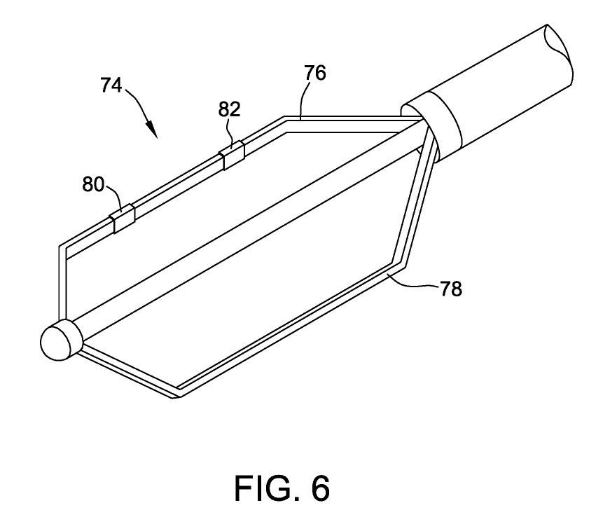

Additional embodiments of electrode baskets within the scope of the present disclosure are shown in FIGS. 6-8. FIG. 6 illustrates a two strut design including asymmetric electrode placement. FIG. 6 shows electrode basket 74 including struts 76 and 78. Strut 76 includes electrodes 80 and 82. Strut 78 does not include electrodes in this embodiment such that the electrodes present are arranged asymmetrically on the struts of the electrode basket.

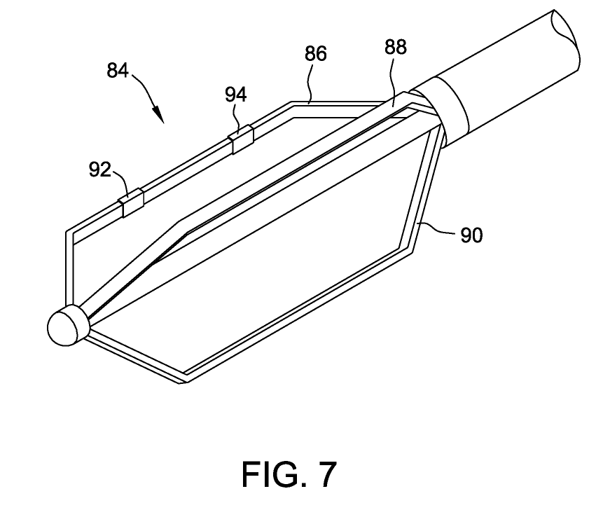

FIG. 7 illustrates a three strut design including asymmetric electrode placement. FIG. 7 shows electrode basket 84 including struts 86, 88, and 90. Strut 86 includes electrodes 92 and 94. Struts 88 and 90 do not include electrodes in this embodiment such that the electrodes present are arranged asymmetrically on the struts of the electrode basket.

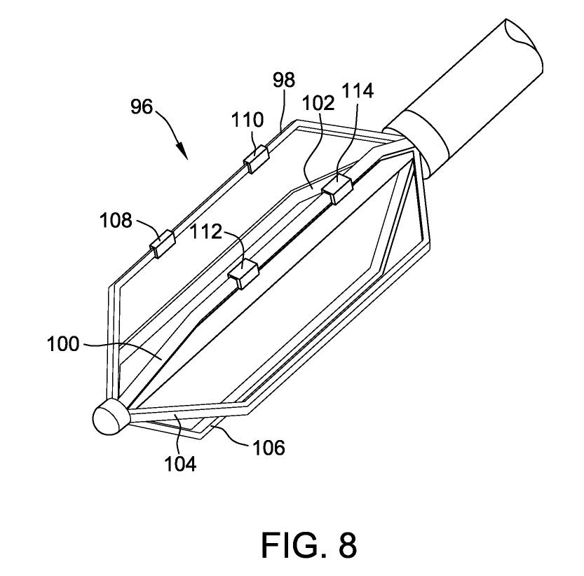

FIG. 8 illustrates a five strut design including asymmetric electrode placement. FIG. 8 shows electrode basket 96 including struts 98, 100, 102, 104, and 106. Strut 98 includes electrodes 108 and 110, and strut 100 includes electrodes 112 and 114. Struts 102, 104, and 106 do not include electrodes in this embodiment such that the electrodes present are arranged asymmetrically on the struts of the electrode basket.

As noted above, the electrodes baskets including asymmetric electrode placement may be utilized in ablation procedures, and specifically renal ablation procedures, to ablate tissue, and specifically tissue within a vessel and reduce the potential for the formation of a circumferential legion merger. The methods generally include the application of energy to the electrode basket two or more separate times during an ablation procedure such that two or more set of ablations, or lesions, are created in the wall of the vessel. In one specific embodiment of the present disclosure, a total of eight lesions is created in the wall of the vessel. In this embodiment, an electrode basket having four struts may be utilized. The electrode basket further includes a total of four electrodes, with two electrodes present on two different, but adjacent, struts. A suitable electrode basket is shown in FIG. 4. In this embodiment, the four strut electrode basket is introduced into the vessel and the four electrodes present are simultaneously energized to create a total of four lesions on the wall of the vessel. Because of the asymmetric distribution of the electrodes on the struts of the electrode basket, the lesions are present on the wall of the vessel in a contiguous angular region of less than 180 degrees; that is, the lesions formed on the wall are present on less than one half of the circumference of the wall of the vessel. With this lesion pattern, the risk of the formation of a continuous circumferential lesion (such as during a spasm of the vessel) is eliminated as at least one half of the circumference of the vessel receives no ablation or lesion creation.

Once the four lesions are created on the vessel wall as described above, in some embodiments the electrode basket may be rotated 180 degrees within the vessel and the process repeated such that an additional four lesions are created in the wall of the vessel. Again, even if the vessel spasms during the creation of the second set of four lesions, because less than one half of the circumference of the vessel wall is being ablated during a single application of energy, the chance for a merger of lesions to form a circumferential lesion is eliminated. Of course, this exemplary embodiment of using a four strut design is one of many method embodiments within the scope of the present disclosure and one skilled in the art based on the disclosure herein will understand how the other disclosed electrode basket designs may be utilized in accordance with the description herein.

Although certain embodiments of this disclosure have been described above with a certain degree of particularity, those skilled in the art could make numerous alterations to the disclosed embodiments without departing from the spirit or scope of this disclosure. All directional references (e.g., upper, lower, upward, downward, left, right, leftward, rightward, top, bottom, above, below, vertical, horizontal, clockwise, and counterclockwise) are only used for identification purposes to aid the reader's understanding of the present disclosure, and do not create limitations, particularly as to the position, orientation, or use of the disclosure. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other. It is intended that all matter contained in the above description or shown in the accompanying drawings shall be interpreted as illustrative only and not limiting. Changes in detail or structure may be made without departing from the spirit of the disclosure as defined in the appended claims.

When introducing elements of the present disclosure or the preferred embodiment(s) thereof, the articles “a”, “an”, “the”, and “said” are intended to mean that there are one or more of the elements. The terms “comprising”, “including”, and “having” are intended to be inclusive and mean that there may be additional elements other than the listed elements.

As various changes could be made in the above constructions without departing from the scope of the disclosure, it is intended that all matter contained in the above description or shown in the accompanying drawings shall be interpreted as illustrative and not in a limiting sense.

1-8. (canceled)

9. A method of ablating vessel tissue, the method comprising:
introducing an electrode basket into the vessel at a first ablation position, wherein the electrode basket comprises a plurality of struts and a plurality of electrodes, wherein each strut of the plurality of struts includes a linear central portion defined by a pair of living hinges, wherein the plurality of electrodes are arranged on a subset of the plurality of struts, and wherein each electrode of the plurality of electrodes is positioned on the central portion of a respective strut of the subset of the plurality struts; simultaneously energizing the plurality of electrodes such that a first contiguous angular region including less than half of a circumference of a wall of the vessel is ablated;
rotating the electrode basket to a second ablation position; and
simultaneously energizing the plurality of electrodes such that a second contiguous angular region different from the first contiguous angular region and including less than half of a circumference of the wall of the tissue is ablated such that lesions in the first contiguous angular region and lesions in the second contiguous angular region do not merge.

10. (canceled)

11. The method of claim 10 wherein rotating the electrode basket to a second ablation position such a second contiguous angular region of less than 180 degrees different than the first contiguous angular region of less than 180 degrees is ablated upon comprises rotating the electrode basket about 180 degrees.

12. The method of claim 9 wherein the plurality of struts comprises four struts.

13. The method of claim 12 wherein the subset of the plurality of struts comprises two struts.

14. The method of claim 13 wherein the two struts each include two electrodes of the plurality of electrodes.

15. The method of claim 14 wherein the two struts are adjacent struts.

16-20. (canceled)

21. A method of ablating vessel tissue, the method comprising:
introducing an electrode basket into the vessel at a first ablation location having a first longitudinal position, wherein the electrode basket comprises a plurality of struts and a plurality of electrodes;
ablating a first contiguous angular region including less than half of a circumference of a wall of the vessel using the plurality of electrodes;
rotating the electrode basket to a second ablation position having the first longitudinal position; and
ablating a second contiguous angular region different from the first contiguous angular region and including less than half of a circumference of the wall of the tissue such that lesions in the first contiguous angular region and lesions in the second contiguous angular region do not merge.

22. The method of claim 21 wherein rotating the electrode basket to a second ablation position comprises rotating the electrode basket about 180 degrees.

23. The method of claim 21 wherein the plurality of struts comprises four struts.

24. The method of claim 23 wherein the plurality of electrodes are arranged on two struts of the four struts.

25. The method of claim 24 wherein the two struts each include two electrodes of the plurality of electrodes.

26. The method of claim 24 wherein the two struts are adjacent struts.
